(19) United States
(12) Patent Application Publication
(10) Pub. No.: US 2024/0138754 A1
(43) Pub. Date: May 2, 2024

(54) Non-Invasive Hydrogen Monitoring to Assess Gut Health and Other Clinical Outcomes

(71) Applicant: University of Cincinnati, Cincinnati, OH (US)

(72) Inventors: Senu Apewokin, Mason, OH (US); William Heineman, Cincinnati, OH (US); Peng Zhang, Cincinnati, OH (US); Sarah Pixley Bell, Cincinnati, OH (US); Michael Smith, Cincinnati, OH (US); Racheal Wilkinson, Wilder, KY (US)

(73) Assignee: University of Cincinnati, Cincinnati, OH (US)

(21) Appl. No.: 18/498,616

(22) Filed: Oct. 31, 2023

Related U.S. Application Data
(60) Provisional application No. 63/420,735, filed on Oct. 31, 2022.

(51) Int. Cl. A61B 5/00; A61B 5/145; A61B 5/1477

(52) U.S. Cl. CPC A61B 5/4238; A61B 5/0015; A61B 5/14546; A61B 5/1477; A61B 5/6832; A61B 5/6838; A61B 5/742

(57) ABSTRACT

A method of assessing gut health in a subject is provided. The method involves placing a device on or near the subject's skin and measuring a level of hydrogen gas emanating through the subject's skin. The device includes an electrochemical sensor capable of detecting hydrogen gas and a means of connecting the device to an apparatus having a central processing unit (CPU). The apparatus provides information on hydrogen gas level and the hydrogen gas level is used to assess gut health of the subject.

### CROSS REFERENCE TO RELATED APPLICATIONS

This application claims priority to U.S. Provisional Application Ser. No. 63/420,735, filed Oct. 31, 2022, which application is hereby incorporated by reference in its entirety.

### STATEMENT REGARDING FEDERALLY SPONSORED RESEARCH OR DEVELOPMENT

This invention was made with government support under 2UL1TR001425-05A1 awarded by the National Institute of Health Clinical and Translation Science Award program. The U.S. Government has certain rights in the invention.

### TECHNICAL FIELD

The present invention relates to a device that measures hydrogen gas emitted from dermal tissue.

### BACKGROUND OF THE INVENTION

Humans do not produce hydrogen gas (H₂). However, many microbes found in the human gut produce hydrogen. The microbes in the gut that use hydrogen in their metabolic pathways impact human nutrition, health, and wellbeing. The hydrogen produced from gut microbiota can have beneficial effects on their human host. But an excess of hydrogen can also have harmful effects on the human host, as it is proposed to be associated with various gastrointestinal disorders, including Irritable Bowel Syndrome, inflammatory bowel disease, and obesity.

The gut microbiome is a complex community with over 10,000 components that contribute to hundreds of pathways. The gut microbiome is a high-dimensional property, which makes evaluating changes that are true drivers of health outcomes very challenging. Dimensionality reduction is critical to overcoming this challenge. Historically, dimensionality reduction has been achieved mainly through bioinformatic approaches. While these approaches may be helpful, they often cannot occur quickly enough to impact care at the bedside, and generation of actionable information is resource intense.

Hydrogen gas produced by the microbes in the gut is expelled from the gut in numerous ways, including being diffused through the intestinal epithelium into the blood and carried to the lungs, which is then expelled as a component of breath. There are currently available techniques to measure hydrogen through breath testing. These breath tests can also detect methane and have established associations with inflammatory bowel disease (IBD). However, there are several drawbacks to these tests, as they require patient cooperation, require a patient to eat or drink, are limited to typically three measurements, no continuous measurements, require skilled expertise for processing results, processing is resource intensive, and the turnaround time (likely greater than one week) for the results is greater than what is useful in acute and intensive care.

Experiments have also found hydrogen gas emanating from the palm. Unlike breath testing, it does not require a patient to eat. However, there are several drawbacks to this method as well. Like breath testing, hand gas testing requires patient cooperation, is typically limited to 3 measurements, and turnaround time (likely greater than one week) for the results is greater than what is useful in acute and intensive care. Further, testing is limited to just the hand and requires gas chromatography mass spectrometry, which is expensive and limited in availability and practicality.

Additionally, clinical trials are underway for an ingestible telemetric gas-sensing capsule for diagnosing gut disorders. In addition to measuring hydrogen in the gut, the capsule also measures oxygen and carbon dioxide. The results are measured in real-time and can connect via Bluetooth to a Bluetooth compatible device, such as a phone. However, like the breath testing and hand gas testing, there are several drawbacks to the capsule. The capsule is likely to be an expensive one-time use test that requires a patient to swallow a large capsule, which may be difficult for some patients. Further, there may be biocompatibility issues, risk of electrical and mechanical safety, and possible interference with other medical devices.

Gut microbial communities have been extensively studied, with several reports documenting a correlation between microbiome profiles and health outcomes. Currently, microbiome studies are costly, time-consuming, and not available at a patient's bedside. A need exists for a rapid, non-invasive way to study the gut microbiome and the changes within, with easily digestible data, less complicated equipment, less complicated testing procedures, and less complicated bioinformatic analysis to advance our understanding of how these changes impact health and produce actionable information before clinical complications at the bedside to improve health outcomes, ideally with a single analyte or marker.

Additionally, hydrogen gas concentration is a critical factor in acid-base homeostasis. Acid-base balance is a biological process necessary for life. Problems in maintaining acid-base balance can be life-threatening and a common problem for patients who have diabetes or who are suffering from heart disease, vomiting, drug toxicity, lung disease, or other issues. Typically, the lungs and kidneys are monitored for acid-base homeostasis, but gut microbiota is not. Although research on the impact of the gut on acid-base balance is limited, it is known that the gut microbiota affects hydrogen (H⁺); thus, it can be presumed that the gut microbiota affects acid-base balance.

There are several current monitoring systems for acid-base balance. Bicarbonate testing provides precise quantitative data in a relatively fast turnaround time. But bicarbonate testing requires a blood draw and cannot be continuously monitored. Arterial blood gas testing is another option to test for acid-base balance. Arterial blood gas testing provides precise quantitative data in a relatively fast turnaround time. However, the test requires a highly complex arterial blood draw, and the specimen has a very short stability. A third option for acid-base balance testing is urinalysis. Urinalysis is non-invasive, as only urine collection is needed, and the test can be performed bedside. But the test provides only qualitative data and has a potential for interference. A need exists for a low complexity, non-invasive acid-base balance test that provides continuous monitoring and instant results.

### SUMMARY OF THE INVENTION

In one embodiment, the present invention involves a method of assessing gut health in a subject. The method involves placing a device on or near the subject's skin and measuring a level of hydrogen gas emanating through the subject's skin. The device includes an electrochemical sensor capable of detecting hydrogen gas and a means of connecting the device to an apparatus having a central processing unit (CPU). The apparatus provides information on hydrogen gas level and the hydrogen gas level is used to assess gut health of the subject.

In one embodiment, the apparatus is selected from the group consisting of computers, smartphones, and tablets. In another embodiment, the apparatus is capable of displaying graphical readings based on information from the sensor. In one embodiment, the device further includes a probe connected to the electrochemical sensor by an electrical wire.

In another embodiment, the electrochemical sensor is capable of providing quantitative hydrogen gas level data. In one embodiment, the electrochemical sensor is capable of providing continuous data regarding the hydrogen gas level. In another embodiment, the device is reusable.

In one embodiment, the device further includes a clip connected to the electrochemical sensor by an electrical wire. In another embodiment, the device is located near an area of the subject's skin selected from the group consisting of sternal notch, belly button, right iliac crest, left iliac crest, finger, and earlobe. In one embodiment, the device further includes a probe in wireless communication with the electrochemical sensor. In another embodiment, the wireless communication is enabled via a wi-fi or bluetooth connection.

In one embodiment, the device further includes a clip in wireless communication with the electrochemical sensor. In another embodiment, the wireless communication is enabled via a wi-fi or bluetooth connection. In one embodiment, the clip includes a flexible device or a device worked by a spring mechanism that grips or clasps connected to an electrochemical sensor. In another embodiment, the device is located near an area of the subject's skin selected from the group consisting of one or more fingers and one or more earlobes. In one embodiment, the electrochemical sensor includes an adhesive portion.

In another embodiment, the present invention is method of assessing gut health in a subject. The method involves placing a device on or near the subject's skin and measuring a level of hydrogen gas emanating through the subject's skin. The device incudes a colorimetric sticker capable of detecting hydrogen gas. The sticker provides information on hydrogen gas level and the hydrogen gas level is used to assess gut health of the subject. In one embodiment, the device is located near an area of the subject's skin selected from the group consisting of sternal notch, belly button, right iliac crest, left iliac crest, finger, and earlobe.

### BRIEF DESCRIPTION OF THE DRAWINGS

The foregoing summary, as well as the following detailed description of preferred embodiments of the application, will be better understood when read in conjunction with the appended drawings.

FIG. 1 is a schematic showing how changes in the relative abundance of bacteria with hydrogenase genes (i.e. the presumed ability to metabolize hydrogen gas) may affect levels of H₂ in the blood.

FIG. 2A is an abdominal x-ray image of a participant, a chemotherapy patient undergoing a bone marrow transplant (BMT), at 15 days from chemotherapy administration, showing small bowel obstruction (SBO).

FIG. 2B is an abdominal x-ray image of the participant at day 15 from chemotherapy start date after a nasogastric tube was placed, showing SBO cleared.

FIG. 3A is a graph showing median topical hydrogen gas measurements from day 8 from chemotherapy start date to day 16 taken from the participant's sternal notch.

FIG. 3B is a graph showing median topical hydrogen gas measurements from day 8 from chemotherapy start date to day 16 taken from the participant's belly button.

FIG. 3C is a graph showing median topical hydrogen gas measurements from day 8 from chemotherapy start date to day 16 taken from the participant's right iliac crest.

FIG. 3D is a graph showing median topical hydrogen gas measurements from day 8 from chemotherapy start date to day 16 taken from the participant's left iliac crest.

FIG. 3E is a graph showing median topical hydrogen gas measurements from day 8 from chemotherapy start date to day 16 taken from the participant's fingertip.

FIG. 3F is a graph showing median topical hydrogen gas measurements from day 8 from chemotherapy start date to day 16 taken from the participant's earlobe.

FIG. 4 is a graph showing the participant's serum bicarbonate measurements through day 16.

FIG. 5 is a graph showing the change in abundance of hydrogenase genes over time in a hydrogen sensor study patient.

FIG. 6 is a chart showing the total number or reads from metagenomic analysis of the relative abundance of hydrogenase genes measured between 1 day from chemotherapy to 19 days from chemotherapy. Alignment is defined as 100% query coverage.

FIG. 7 is a schematic showing the timeline for H₂ measurements taken from mice given antibiotics.

FIG. 8A is a graph showing H₂ levels in both control mice and mice given antibiotics. The graph shows H₂ levels prior to dosing with antibiotics.

FIG. 8B is a graph showing H₂ levels in both control mice and mice given antibiotics. The graph shows H₂ levels a day after dosing with antibiotics.

FIG. 8C is a graph showing H₂ levels in both control mice and mice given antibiotics. The graph shows H₂ levels three days after dosing with antibiotics.

FIG. 9 is a graph showing the effect of hydrogen gas on pH.

FIG. 10 is a graph showing in-vitro bubbling studies using 30 mls of whole blood.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “central processing unit” or “CPU” means a processor having a control unit and an arithmetic logic unit.

As used herein, “gut health” means the overall functioning of an individual's GI tract, and may include factors such as digestion and absorption of food, the presence of GI illness, the presence of normal and stable intestinal microflora and immune status.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present invention is a method of assessing gut health in a subject. The method involves placing a device on or near the subject's skin and measuring a level of hydrogen gas emanating through the subject's skin. The device includes an electrochemical sensor capable of detecting hydrogen gas and a means of connecting the device to an apparatus having a central processing unit (CPU). The apparatus provides information on hydrogen gas level and the hydrogen gas level is used to assess gut health of the subject.

The present invention involves the discovery that a by-product, such as a metabolite, of the gut microbial community that impacts host biology can be a reliable surrogate for detecting microbial community profiles and the changes that occur within. Such a surrogate can be utilized as a “vital sign” of gut health, as is done with oxygen saturation to monitor cardiopulmonary function. Hydrogen gas is a promising candidate that satisfies these requirements.

Historically, the respiratory and renal systems have been recognized as the major organ systems credited with maintaining acid-base balance, given their pivotal compensatory mechanisms. Even though the gastrointestinal tract may have a less prominent role in compensatory processes, it still impacts acid-base balance overall, given its ability to generate hydrogen gas and protons. In human acid-base physiology, particularly anion gap metabolic acidosis, there is a contribution to the acidity that is not accounted for by the concentration of inorganic compounds and thus attributed to organic compound contributions and/or gastrointestinal losses. Gut hydrogen may contribute significantly to this acid-base homeostasis.

Hydrogen gas has been found to emanate from human skin. As free hydrogen can be measured through dermal tissue, the present invention uses topical, non-invasive monitoring of hydrogen as a surrogate marker for gut microbial profiles and the changes that occur within. Similar to the use of pulse oximeters to assess cardiopulmonary status, topical non-invasive hydrogen monitoring, also referred to as “GUT-HY,” is used to assess gut health.

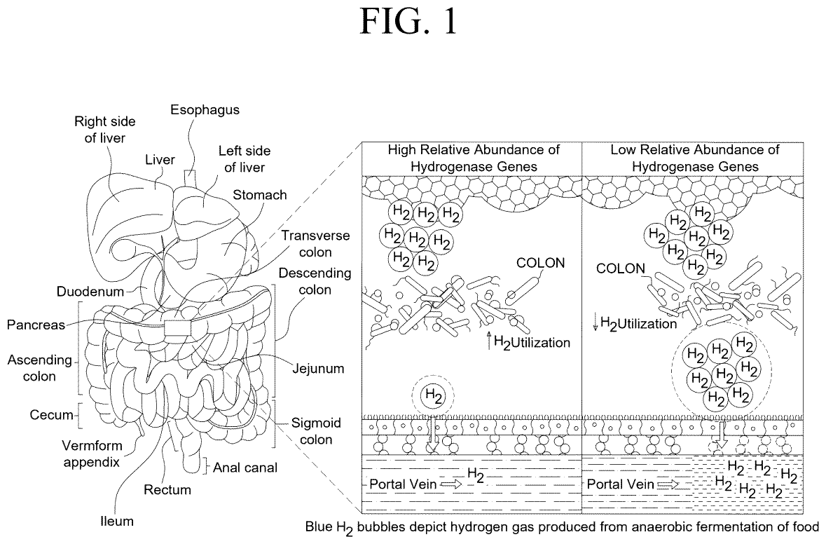

The present invention is a topical non-invasive hydrogen monitoring system that can assess gut microbiome-driven metabolic changes and measure hydrogen gas which can be used to predict outcomes related to or dependent upon acid-base balance. The physiologic range of blood pH of 7.35 to 7.45 is very narrow yet derangements have adverse health implications. Acidosis (levels below 7.35) and alkalosis (levels above 7.45) have been associated with a wide variety of health impairments. The abundance of many inorganic substances such as CO₂, HCO₃ affects acid-base balance. Our data suggests hydrogen affects acid-base also. Humans do not produce hydrogen gas; however, bacteria in the gastrointestinal tract use an excess of hydrogen gas, as shown in FIG. 1. This non-invasive hydrogen monitoring system has the potential to measure dysbiosis in real-time by a change in hydrogen concentrations, which is a critical factor in acid-base homeostasis. Current acid-base testing methods are invasive, as they typically require blood draws, and cannot be continuously monitored. The present invention provides continuous monitoring, is non-invasive, and provides instant results with low complexity testing. In some embodiments of the present invention, the monitoring device may use wires and bulky equipment.

There are several advantages to the present invention, which uses non-invasive hydrogen measurement as a “vital sign.” The present invention uses a single marker, and the data is easily digested, as the data is limited to monitoring hydrogen gas. Readings are in real-time, which provides quicker analysis over other hydrogen gas measurement methods. The equipment is less complicated in that the invention uses the topical placement of the measuring device, such as a sensor, probe, or adhesive. The testing procedures of the present invention are less complicated, as there is no need for PCR or cultures for initial monitoring. The present invention provides less complex bioinformatic analysis since it is not necessary to detect the presence of a change. The goal of the present invention is to identify a change before clinical complications arise.

For the present invention, various monitoring devices can be used for topically measuring hydrogen gas. In one embodiment of the present invention, the measuring device is an electrochemical probe. The invention can be further described as a probe connected to an electrochemical sensor used to detect and measure the hydrogen gas emanating through a person's skin. The electrochemical probe can provide quantitative data, continuous reading, may be reusable, and color analysis and bias are not a factor. In some embodiments, the device may require the use of wires, require placement of the probe, may need additional equipment, and the readings could be variable due to movements. The device may be connected to an apparatus, such as a computer, smartphone, tablet, or other devices used to display the real-time graphical readings from the measuring device. In some embodiments, the measuring device can be connected to a computer via wires, wireless, such as over a Wi-Fi connection, or Bluetooth.

In another embodiment of the present invention, the measuring device is an electrochemical clip. This embodiment of the invention can be further described as a flexible device or a device worked by a spring mechanism that grips or clasps connected to an electrochemical sensor. In certain embodiments, this device will provide quantitative data, continuous readings, be reusable, and color analysis and bias are not a factor. In the clip embodiment, the areas for the readings are limited to areas of the body that can be clipped, such as the finger or earlobe, and may require wires.

In another embodiment of the present invention, the measuring device is an electrochemical sticker. This embodiment of the invention can be further described as an adhesive portion of the device which is placed topically on a person's body and an electrochemical sensor to detect and measure the hydrogen gas emanating through the person's skin. The electrochemical sticker provides easy accessibility, quantitative data, and a continuous reading without color analysis and bias being a factor. In this sticker embodiment, the electrochemical sticker is typically for one-time use. In some embodiments, the sticker may need the use of wires.

In another embodiment of the present invention, the measuring device is a colorimetric sticker. In this embodiment, an adhesive portion of the device is placed topically on a person's body and a colorimetric sensor is used to detect hydrogen gas. This device provides easy accessibility but would not likely be able to provide continuous readings and the quantitative data may be limited.

Various locations on a person's body can be used to place the monitoring device or sensors and are not limited to the areas mentioned herein. In different embodiments, areas to place the monitoring device or sensor include the sternal notch, belly button, right iliac crest, left iliac crest, finger, and earlobe. A sensor can be placed on any area where a portal vein is located, such as the sternal notch. Further, a sensor can be placed on any area in close proximity to the gut, such as the belly button, right iliac crest, and left iliac crest. Additionally, a sensor can be placed at any place on the body that is convenient, such as the finger and earlobe.

Results from the present invention can be monitored in numerous ways, including detection of changes over time, such as delta checks and moving averages, by monitoring trends, analyzing the quantitative data, or, in one embodiment, by real-time continuous graphical reads. Real-time continuous graphical reads would not need additional steps to obtain actionable information. When compared to the breath test, the present invention has several advantages. The present invention provides continuous reads, where the breath test does not. The present invention does not require a person to eat or drink before monitoring begins, whereas it is a requirement for the breath test. The present invention can monitor changes over time and detect more than changes due to eating, drugs, physiological changes, and the like. The breath test is a very resource-intensive process where skilled expertise is required for processing. In contrast, the present invention would not be a resource-intensive process, and no skilled expertise is required for processing. Furthermore, the breath test has a long turnaround time for results, where the present invention would provide instant results.

Based on the results of the present invention, treatment steps would be based on the clinician's discretion upon evaluation of the complex and unique patient presentation.

### EXAMPLES

### Example 1

A study was conducted to assess the feasibility of non-invasive hydrogen measurements in patients undergoing chemotherapy and correlate topical hydrogen concentration with changes in the relative abundance of hydrogenotrophic bacteria during chemotherapy. In the study, we collected topical hydrogen gas and serum bicarbonate data, data on the relative abundance of hydrogenase genes with metagenomic analysis, and clinical status data, including infection, digestive health, medications, and other complications.

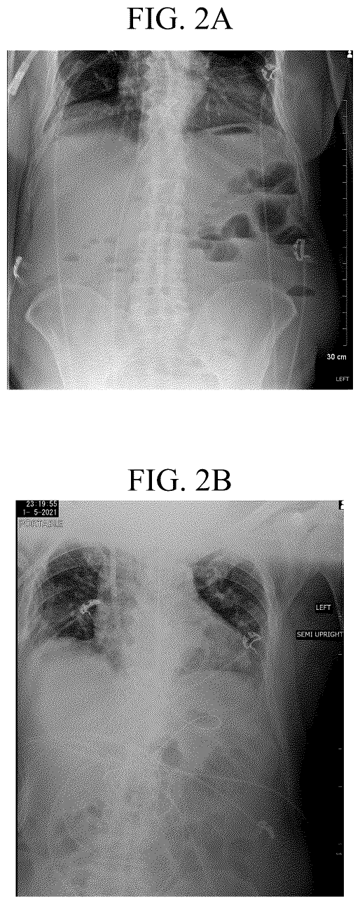

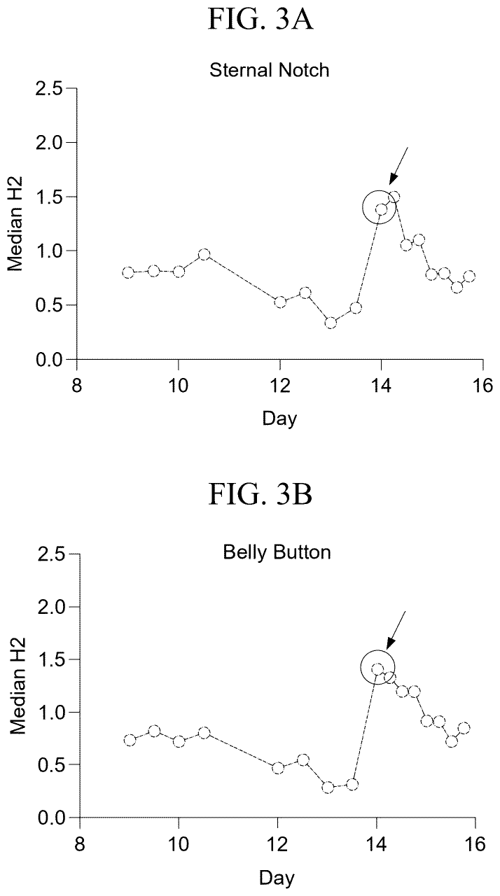

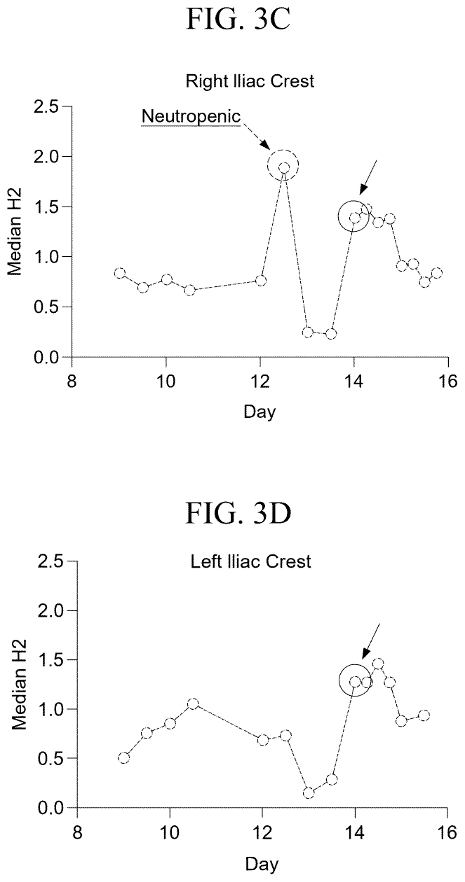

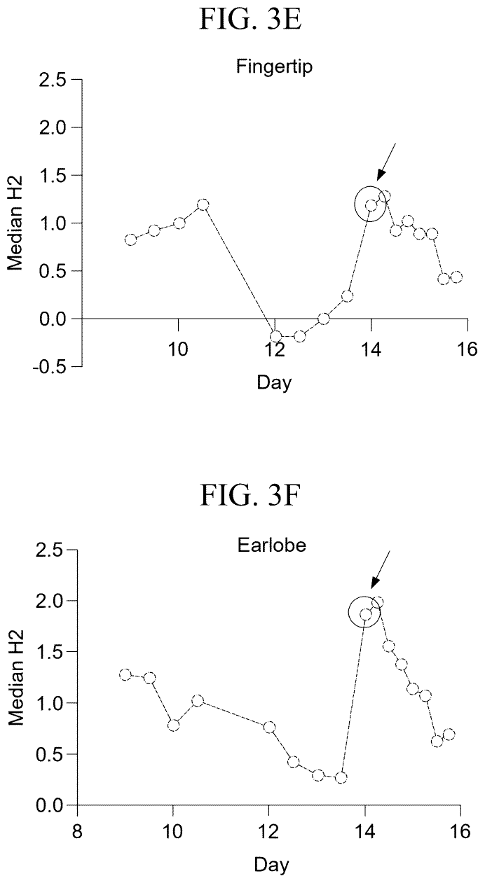

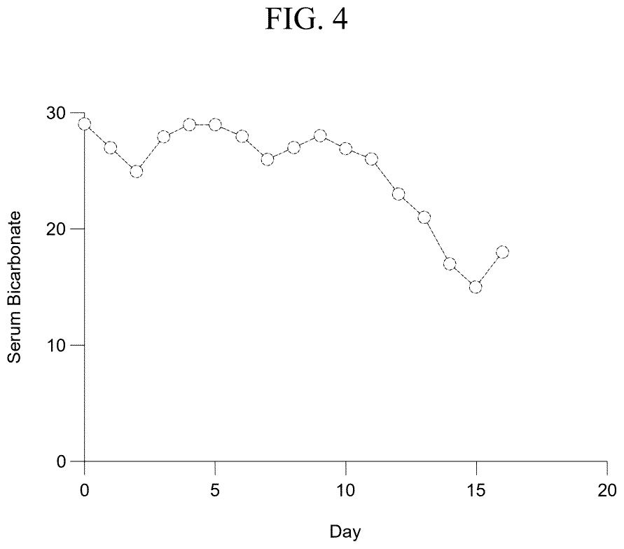

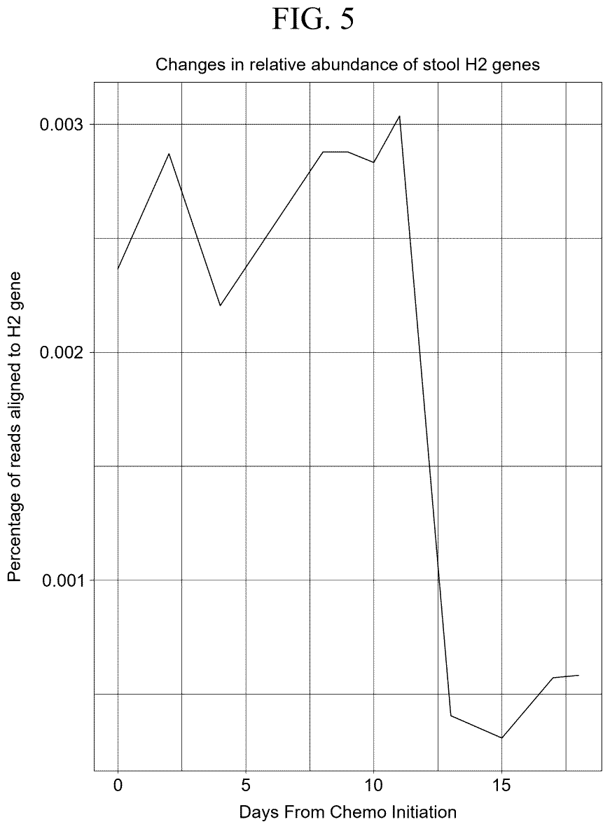

The study was performed on a chemotherapy patient undergoing BMT. The participant was placed on antibiotics beginning on day nine. On day ten, the participant was given proton pump inhibitors. On day twelve, neutropenia and mild SBO was identified. On day fifteen, the participant had significant SBO, as shown in FIG. 2A. That same day, a nasogastric tube was placed, and SBO cleared, as shown in FIG. 2B. FIGS. 3A-3F show topical hydrogen gas findings measured from various locations on the participant between days eight and sixteen. FIG. 4 shows the serum bicarbonate findings from the participant for the duration of the study. FIG. 5 is a graph showing the change in the abundance of hydrogenase genes over time in this hydrogen sensor study patient.

### Example 2

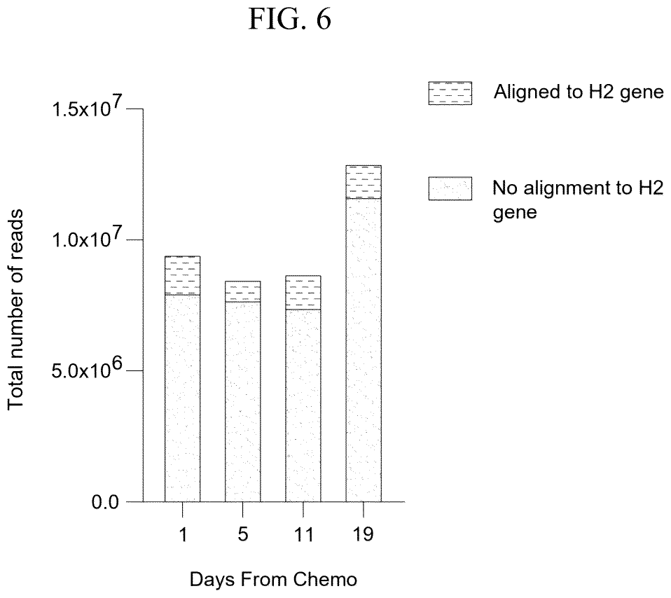

A study was performed on a different chemotherapy patient than the one discussed above. This patient underwent a comparable chemotherapy treatment/BMT to the patient with the hydrogen measurements. FIG. 6 shows the relative abundance of hydrogenase genes from the metagenomic analysis of this different patient undergoing a comparative treatment process.

From the study, we saw three trends. The first trend was an increase in topical hydrogen gas readings with the onset of SBO, as shown in FIGS. 3A-3F. The second trend was a decrease in serum bicarbonate levels with the onset of SBO, as shown in FIG. 4. Finally, the third trend was a decrease in the relative abundance of hydrogenase genes days after chemotherapy, as shown in FIG. 6.

### Example 3

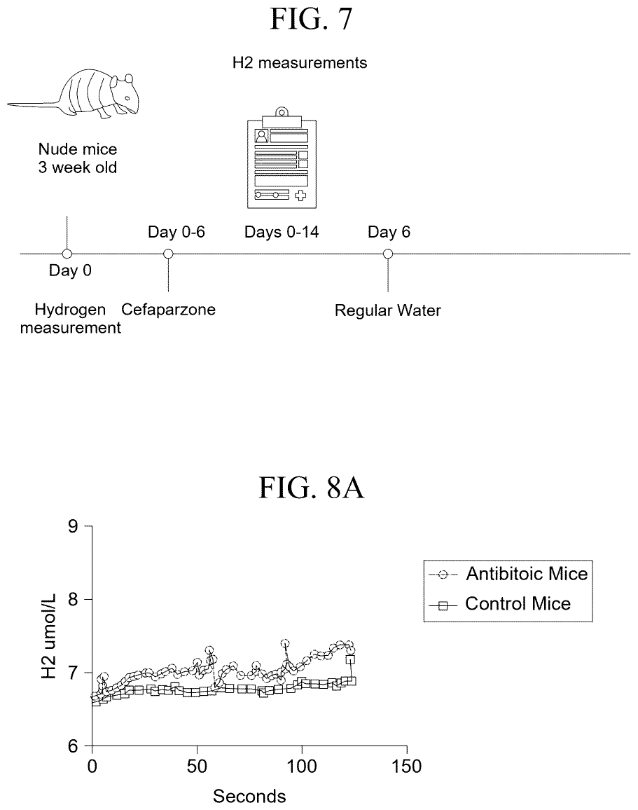

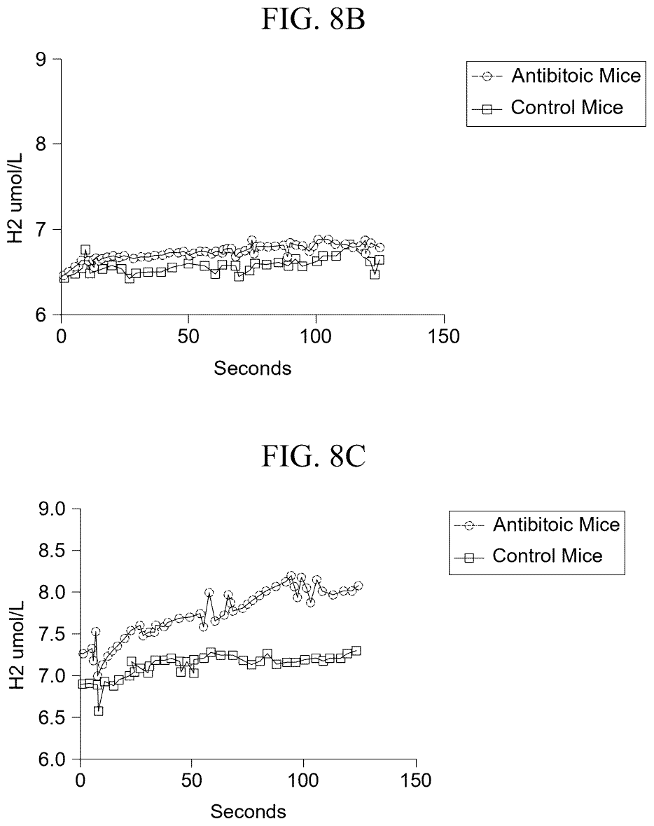

H₂ levels were measured over time for mice given an antibiotic (Cefaparzone) and a control group of mice that did not receive antibiotics (see FIG. 7). 3-week old nude (hairless) mice (four per cage) were exposed to cefoparazone (antibiotic) in drinking water for 7 days. As a control, a set of mice were exposed to sucrose only. Hydrogen measurements were performed prior to antibiotics, day of antibiotic initiation and 3 days after antibiotics initiation. Hydrogen measurements were performed by placing the sensor probe onto the abdomen of nude mice. FIG. 8A shows H₂ levels in both the control mice and the mice that would receive antibiotics. The data presented in FIG. 8A shows H₂ levels prior to dosing with antibiotics. FIG. 8B shows H₂ levels for both groups of mice a day after dosing with antibiotics. FIG. 8C shows H₂ levels for both groups of mice three days after dosing with antibiotics.

### Example 4

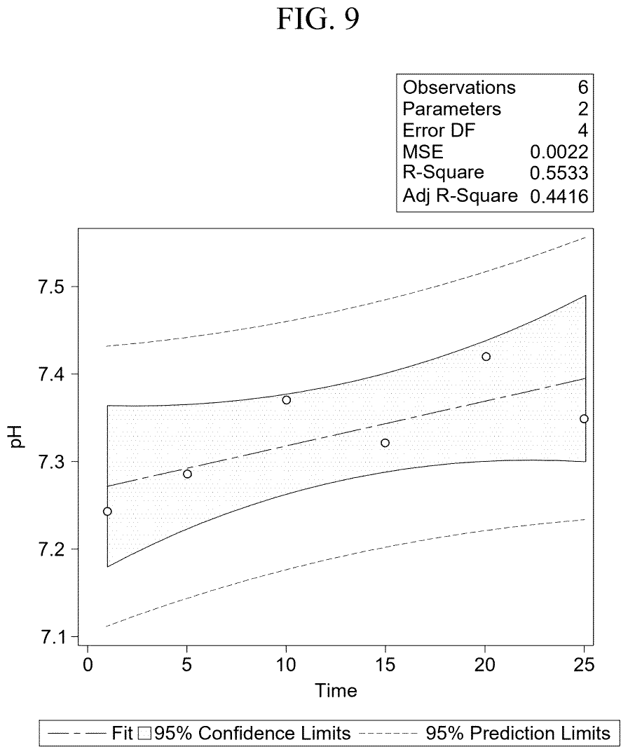

To assess the effect of hydrogen gas on pH, hydrogen gas was blown through whole blood and pH was measured at five-minute intervals. 30 mls of whole blood was collected from a healthy volunteer, placed in a v-bottom vessel, and sealed with a plastic adhesive. Hydrogen gas was then bubbled through the blood and a capillary tube was used to collect 60 microliters of blood at 10-minute intervals for pH measurement. pH measurements were performed with an iSTAT instrument (Abbott). The results are shown in FIG. 9.

### Example 5

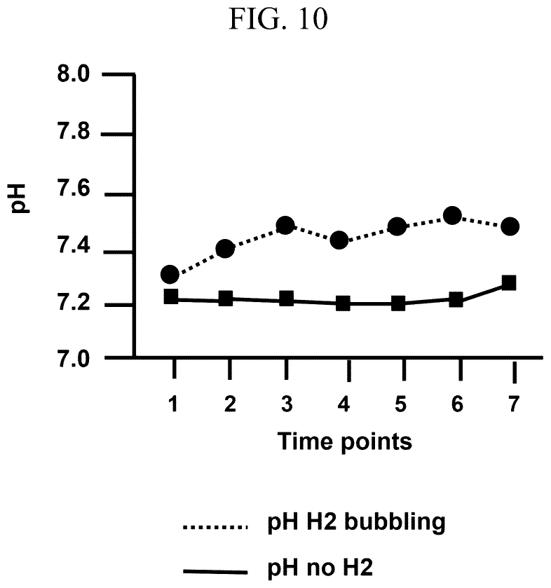

30 mls of whole blood was collected from a healthy volunteer, placed in a v-bottom vessel, and sealed with a plastic adhesive. Hydrogen gas was then bubbled through the blood and a capillary tube was used to collect 60 microliters of blood at 10-minute intervals for pH measurements. pH measurements were performed with an iSTAT instrument (Abbott). The results are shown in FIG. 10 as the dotted lines. As a control, 30 mls of blood were also placed in an identical vessel and measured pH at 10-minute intervals (with no bubbling) shown as the solid line.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of assessing gut health in a subject comprising placing a device on or near the subject's skin and measuring a level of hydrogen gas emanating through the subject's skin, wherein the device comprises an electrochemical sensor capable of detecting hydrogen gas and a means of connecting the device to an apparatus comprising a central processing unit (CPU); and further, wherein the apparatus provides information on hydrogen gas level and the hydrogen gas level is used to assess gut health of the subject.

2. The method of claim 1 wherein the apparatus is selected from the group consisting of computers, smartphones, and tablets.

3. The method of claim 1 wherein the apparatus is capable of displaying graphical readings based on information from the sensor.

4. The method of claim 1 wherein the device further comprises a probe connected to the electrochemical sensor by an electrical wire.

5. The method of claim 1 wherein the electrochemical sensor is capable of providing quantitative hydrogen gas level data.

6. The method of claim 5 wherein the electrochemical sensor is capable of providing continuous data regarding the hydrogen gas level.

7. The method of claim 1 wherein the device is reusable.

8. The method of claim 1 wherein the device further comprises a clip connected to the electrochemical sensor by an electrical wire.

9. The method of claim 1 wherein the device is located near an area of the subject's skin selected from the group consisting of sternal notch, belly button, right iliac crest, left iliac crest, finger, and earlobe.

10. The method of claim 1 wherein the device further comprises a probe in wireless communication with the electrochemical sensor.

11. The method of claim 10 wherein the wireless communication is enabled via a wi-fi or bluetooth connection.

12. The method of claim 1 wherein the device further comprises a clip in wireless communication with the electrochemical sensor.

13. The method of claim 12 wherein the wireless communication is enabled via a wi-fi or bluetooth connection.

14. The method of claim 8 wherein the clip comprises a flexible device or a device worked by a spring mechanism that grips or clasps connected to an electrochemical sensor.

15. The method of claim 8 wherein the device is located near an area of the subject's skin selected from the group consisting of one or more fingers and one or more earlobes.

16. The method of claim 1 wherein the electrochemical sensor comprises an adhesive portion.

17. A method of assessing gut health in a subject comprising placing a device on or near the subject's skin and measuring a level of hydrogen gas emanating through the subject's skin, wherein the device comprises a colorimetric sticker capable of detecting hydrogen gas, wherein the sticker provides information on hydrogen gas level and the hydrogen gas level is used to assess gut health of the subject.

18. The method of claim 17 wherein the device is located near an area of the subject's skin selected from the group consisting of sternal notch, belly button, right iliac crest, left iliac crest, finger, and earlobe.
